(19) United States
(12) Patent Application Publication
(10) Pub. No.: US 2026/0298905 A1
(43) Pub. Date: Oct. 1, 2026

(54) SENSOR, CONTROL DEVICE, MEASUREMENT SYSTEM, CONTROL METHOD OF SENSOR, AND CONTROL METHOD OF CONTROL DEVICE

(71) Applicant: SONY GROUP CORPORATION, TOKYO (JP)

(72) Inventor: TAKAHIRO OISHI, KANAGAWA (JP)

(21) Appl. No.: 19/489,898

(22) PCT Filed: May 7, 2024
(86) PCT No.: PCT/JP2024/016980

§ 371 Date: Dec. 4, 2025

(30) Foreign Application Priority Data
Jun. 28, 2023 (JP) 2023-105872

(51) Int. Cl. G01N 33/24; A01G 27/00

(52) U.S. Cl. CPC G01N 33/246; A01G 27/00

(57) ABSTRACT

In a system that measures a parameter related to moisture of soil, power consumption is reduced. A sensor includes a measurement section and a control unit. In the sensor including the measurement section and the control unit, the measurement section measures the parameter related to the moisture of the soil. Furthermore, in the sensor including the measurement section and the control unit, the control unit controls a measurement frequency of the parameter within a first measurement period immediately before an irrigation timing to the soil to a value higher than a predetermined value, and controls the measurement frequency before the first measurement period to a value lower than the predetermined value.

### TECHNICAL FIELD

The present technology relates to a sensor. More specifically, the present technology relates to a sensor, a control device, a measurement system, a control method of a sensor, and a control method of a control device which are for measuring a water content, a water potential, and the like.

### BACKGROUND ART

In the related art, in an agricultural field, it has been required to measure a parameter such as a water content or a water potential of soil for the healthy growth of crops, and to perform irrigation at an appropriate frequency and timing on the basis of measurement values. For example, a system has been proposed in which, in a state where a plurality of moisture sensors for measuring a water content is disposed in a farm field, the measurement frequency of the water content is reduced as the distance between the moisture sensor and the crop increases (for example, refer to Patent Document 1).

### CITATION LIST

### Patent Document

Patent Document 1: Japanese Patent Application Laid-Open No.

### SUMMARY OF THE INVENTION

### Problems to be Solved by the Invention

In the above-described related art, a reduction in power consumption of the system is attempted by lowering the measurement frequency as the distance between the moisture sensor and the crop increases. However, in the above-described related art, it is difficult to further reduce the power consumption.

The present technology has been made in view of such a situation, and an object thereof is to reduce power consumption in a system for measuring a parameter related to moisture of soil.

### Solutions to Problems

The present technology has been made to solve the above-described problems, and a first aspect thereof is a sensor and a control method thereof, the sensor including a measurement section that measures a parameter related to moisture of soil; and a control unit that controls a measurement frequency of the parameter within a first measurement period immediately before an irrigation timing to the soil to a value higher than a predetermined value, and controls the measurement frequency before the first measurement period to a value lower than the predetermined value. Therefore, an effect of reducing power consumption of the sensor is brought about.

Furthermore, in the first aspect, a communication unit that receives predetermined setting information may be further provided. Therefore, an effect of allowing setting information to be set in the sensor is brought about.

Furthermore, in the first aspect, the setting information may include the irrigation timing, and the first measurement period may be a period from a timing, which is a predetermined time before the irrigation timing, to the irrigation timing. Therefore, an effect of allowing measurement to be performed on the basis of the set irrigation timing is brought about.

Furthermore, in the first aspect, the setting information may include a predetermined measurement threshold value, and the first measurement period may be a period from a timing at which the parameter less than the measurement threshold value is measured to the irrigation timing. Therefore, an effect of allowing measurement to be performed on the basis of the set measurement threshold value is brought about.

Furthermore, in the first aspect, the setting information may include information indicating whether an installation place of the sensor is indoors or outdoors. Therefore, an effect of allowing control to be performed according to an installation place is brought about.

Furthermore, in the first aspect the control unit may control the measurement frequency within a second measurement period immediately after the irrigation timing to a value higher than the predetermined value, and control the measurement frequency within a period that corresponds to neither the first measurement period nor the second measurement period to a value lower than the predetermined value. Therefore, an effect of improving user convenience is brought about.

Furthermore, in the first aspect, the control unit may control the measurement frequency within a third measurement period immediately after rainfall to a value higher than the predetermined value, and control the measurement frequency within a period that corresponds to none of the first measurement period, the second measurement period, and the third measurement period to a value lower than the predetermined value. Therefore, an effect of improving user convenience is brought about.

Furthermore, in the first aspect, the parameter may be a water content. Therefore, an effect of allowing a water content to be measured is brought about.

Furthermore, in the first aspect, the parameter may be a water potential. Therefore, an effect of allowing a water potential to be measured is brought about.

Furthermore, a second aspect of the present technology is a control device and a control method thereof, the control device including: a communication unit that transmits a control signal to a sensor that measures a parameter related to moisture of soil; and a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value. Therefore, an effect of reducing power consumption of the sensor is brought about.

Furthermore, in the second aspect, the parameter may include a water content and a water potential, the sensor may include a moisture sensor that measures the water content and a water potential sensor that measures the water potential, and the control unit may cause the moisture sensor to measure a new water content in a case where the measured water content exceeds a predetermined switching threshold value, and cause the water potential sensor to measure the water potential in a case where the measured water content does not exceed the switching threshold value. Therefore, an effect of allowing switching to an appropriate sensor according to a water content is brought about.

Furthermore, a third aspect of the present technology is a measurement system including: a sensor that measures a parameter related to moisture of soil; and a control device that includes a communication unit that transmits a control signal to the sensor, and a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value. Therefore, an effect of reducing power consumption of the measurement system is brought about.

Furthermore, a fourth aspect of the present technology is a sensor and a control method thereof, the sensor including: a measurement section that measures a parameter related to moisture of soil; and a control unit that controls measurement accuracy of the parameter on the basis of predetermined setting information. Therefore, an effect of reducing power consumption of the sensor is brought about.

### BRIEF DESCRIPTION OF DRAWINGS

FIG. 1 is a block diagram illustrating a configuration example of a measurement system in a first embodiment of the present technology.

FIG. 2 is a block diagram illustrating a configuration example of a measurement system when a sensor in the first embodiment of the present technology is changed.

FIG. 3 is a block diagram illustrating a configuration example of a user terminal in the first embodiment of the present technology.

FIG. 4 is a diagram for describing setting contents in the first embodiment of the present technology.

FIG. 5 is a block diagram illustrating a configuration example of a moisture sensor in the first embodiment of the present technology.

FIG. 6 is a diagram for describing a measurement method of the moisture sensor in the first embodiment of the present technology.

FIG. 7 is a flowchart illustrating an example of an operation of the user terminal in the first embodiment of the present technology.

FIG. 8 is an example of a setting screen for irrigation time in the first embodiment of the present technology.

FIG. 9 is an example of a setting screen for a threshold value of a water content in the first embodiment of the present technology.

FIG. 10 is an example of a setting screen for a measurement interval and a measurement time in the first embodiment of the present technology.

FIG. 11 is an example of a display screen for a remaining battery level in the first embodiment of the present technology.

FIG. 12 is a graph illustrating an example of a measurement method in a case where water is supplied indoors in the first embodiment of the present technology.

FIG. 13 is a graph illustrating another example of a measurement method in a case where water is supplied indoors in the first embodiment of the present technology.

FIG. 14 is a graph illustrating an example of a measurement method in a case where water is supplied outdoors in the first embodiment of the present technology.

FIG. 15 is a graph illustrating another example of a measurement method in a case where water is supplied outdoors in the first embodiment of the present technology.

FIG. 16 is a block diagram illustrating a configuration example of a measurement system in a modified example of the first embodiment of the present technology.

FIG. 17 is a block diagram illustrating a configuration example of a control device in the modified example of the first embodiment of the present technology.

FIG. 18 is a block diagram illustrating a configuration example of a measurement system in a second embodiment of the present technology.

FIG. 19 is an example of a setting screen for a measurement interval and a measurement time immediately before irrigation in the second embodiment of the present technology.

FIG. 20 is an example of a setting screen for a measurement interval and a measurement time immediately after irrigation in the second embodiment of the present technology.

FIG. 21 is a graph illustrating an example of a measurement method in a case where water is supplied indoors in the second embodiment of the present technology.

FIG. 22 is a graph illustrating another example of a measurement method in a case where water is supplied indoors in the second embodiment of the present technology.

FIG. 23 is a graph illustrating an example of a measurement method in a case where water is supplied outdoors in the second embodiment of the present technology.

FIG. 24 is a graph illustrating another example of a measurement method in a case where water is supplied outdoors in the second embodiment of the present technology.

FIG. 25 is a diagram for describing setting contents in a third embodiment of the present technology.

FIG. 26 is a block diagram illustrating a configuration example of a measurement system in a fourth embodiment of the present technology.

FIG. 27 is a flowchart illustrating an example of an operation of a control device in the fourth embodiment of the present technology.

### MODE FOR CARRYING OUT THE INVENTION

Hereinafter, modes for carrying out the present technology (hereinafter, referred to as embodiments) will be described. The description will be given in the following order.

1. First Embodiment (Example in Which Measurement Frequency Is Increased Immediately Before Irrigation)
2. Second Embodiment (Example in Which Measurement Frequency Is Increased Immediately Before Irrigation and Immediately After Irrigation)
3. Third Embodiment (Example in Which Measurement Accuracy Is Controlled)
4. Fourth Embodiment (Example in Which Sensors Are Switched and Measurement Frequency Is Increased Immediately Before Irrigation)

### 1. First Embodiment

### Configuration Example of Sensor Control System

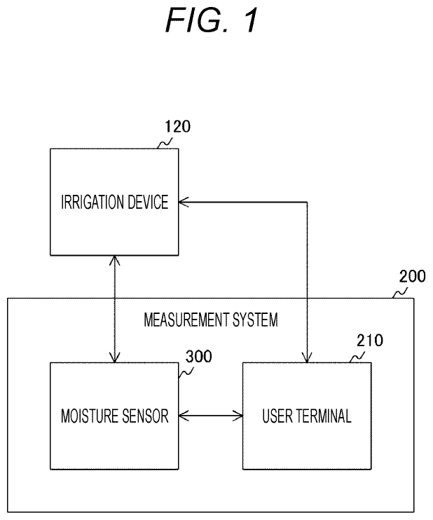

FIG. 1 is a block diagram illustrating a configuration example of a measurement system 200 in a first embodiment of the present technology. The measurement system 200 is for measuring a parameter (such as a water content) related to moisture of soil, and includes a user terminal 210 and a moisture sensor 300. The moisture sensor 300 is installed in soil such as a farm field and a plastic greenhouse.

The user terminal 210 is a terminal for performing various settings. The user terminal 210 can access the moisture sensor 300 and an irrigation device 120 in a wired or wireless manner. The user terminal 210 generates setting information indicating setting contents in accordance with an operation by a user, and transmits the setting information to the moisture sensor 300 and the irrigation device 120. As the user terminal 210, for example, a smartphone or a personal computer is used.

The moisture sensor 300 is for measuring a water content of soil. For example, a volumetric water content representing the water content is measured, and percent is used as a unit of the measurement value. Furthermore, the moisture sensor 300 receives setting information from the user terminal 210. Then, the moisture sensor 300 controls a measurement frequency of the water content within a measurement period immediately before irrigation timing to a value higher than a predetermined value, on the basis of the setting information, and controls the measurement frequency before and after the period to a value lower than the predetermined value. The moisture sensor 300 transmits measurement information indicating a measurement result to the irrigation device 120 and the user terminal 210. Note that the moisture sensor 300 is an example of the sensor described in the claims.

The irrigation device 120 is for performing irrigation on soil in which the moisture sensor 300 is installed. The irrigation device 120 receives setting information from the user terminal 210, and performs irrigation in accordance with the setting information. Furthermore, the irrigation device 120 also changes a next irrigation amount in accordance with a measurement result when the irrigation device 120 receives measurement information from the moisture sensor 300. For example, the irrigation amount is controlled to a smaller value as the measured water content is larger. As the irrigation device 120, a sprinkler or an irrigation hose is used.

Note that the number of moisture sensors 300 in the measurement system 200 is optional, and two or more moisture sensors 300 may be disposed. The number of irrigation devices 120 is also optional, and two or more irrigation devices 120 may be disposed.

Furthermore, the measurement system 200 measures a water content by the moisture sensor 300, but is not limited to this configuration.

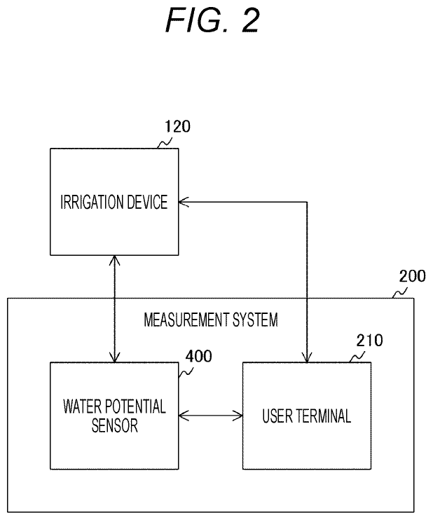

For example, as illustrated in FIG. 2, a water potential sensor 400 may be disposed instead of the moisture sensor 300, and a water potential representing a water stress of soil may be measured instead of the water content. The same applies also to a modified example of the first embodiment and subsequent embodiments.

As the water potential sensor 400, for example, a tensiometer or a sensor in which a porous body is loaded on a dielectric constant measurement sensor is used. As units of the water potential, kilopascals (kPa) or pF in which the value is logarithmically expressed are used.

In general, the water potential sensor 400 can measure values in a dry region with a wide dynamic range and a high resolution, as compared with the moisture sensor 300.

### Configuration Example of User Terminal

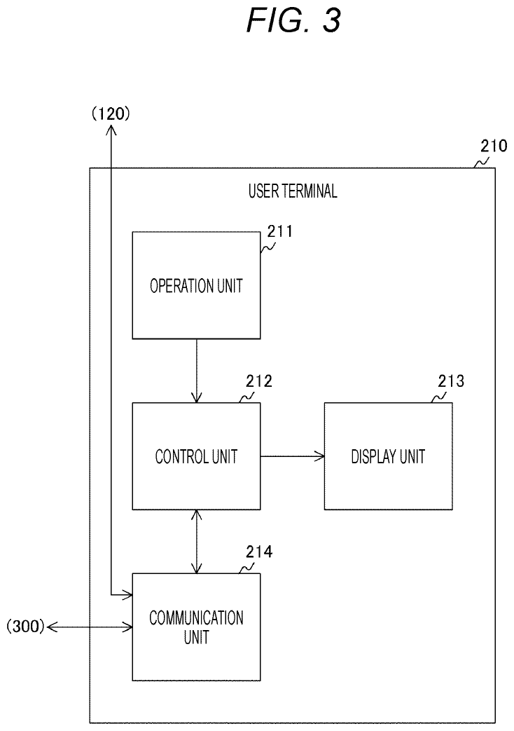

FIG. 3 is a block diagram illustrating a configuration example of the user terminal 210 in the first embodiment of the present technology. The user terminal 210 includes an operation unit 211, a control unit 212, a display unit 213, and a communication unit 214.

The operation unit 211 generates an operation signal in accordance with an operation by the user. The control unit 212 controls the entire user terminal 210. The control unit 212 generates setting information in accordance with the operation signal, and transmits the setting information to the moisture sensor 300 and the irrigation device 120 via the communication unit 214. Details of the setting information will be described later.

The display unit 213 displays various setting screens and the like for performing settings. The communication unit 214 transmits and receives various kinds of information such as setting information to and from devices and equipment such as the moisture sensor 300 and the irrigation device 120 in a wired or wireless manner.

Note that the user performs settings using the user terminal 210, but the present technology is not limited to this configuration. The user may input setting information directly to the moisture sensor 300 without using the user terminal 210 when performing the settings.

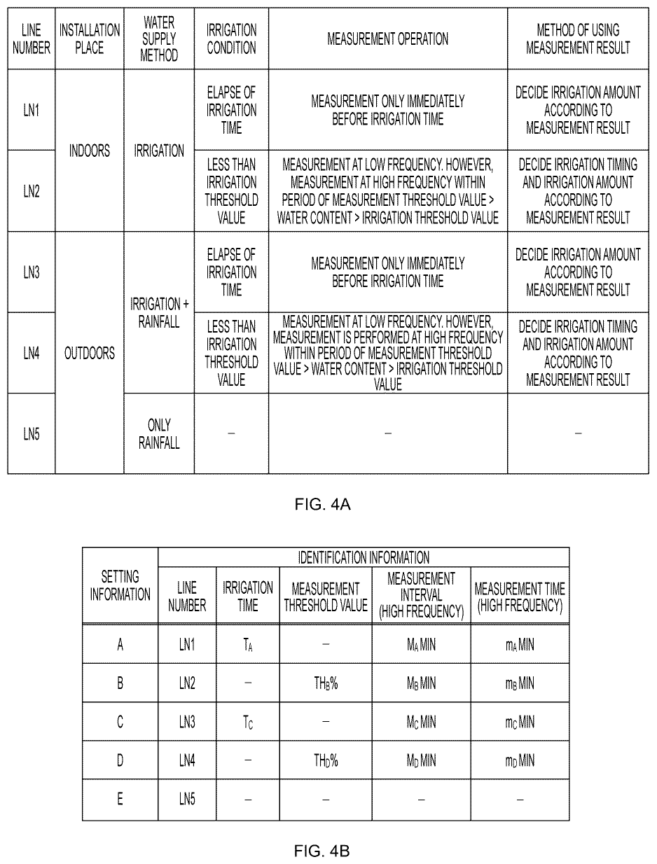

FIG. 4 is a diagram for describing setting contents in the first embodiment of the present technology. In FIG. 4, a is a diagram in which cultivation methods are classified. In FIG. 4, b is a diagram illustrating an example of setting information.

As illustrated in a in FIG. 4, cultivation methods are classified into, for example, five categories. Each cultivation method is assigned with line numbers LN1 to LN5.

In line number LN1, an installation place (in other words, a cultivation place) of the moisture sensor 300 and the irrigation device 120 is indoors, and irrigation is performed by the irrigation device 120. Furthermore, the irrigation device 120 performs irrigation when an irrigation time set by the user has elapsed. Furthermore, the moisture sensor 300 performs measurement of a water content at regular intervals only within the measurement period immediately before the irrigation time. The irrigation device 120 decides a next irrigation amount in accordance with the measurement result.

In line number LN2 as well, an installation place (a cultivation place) of the moisture sensor 300 and the like is indoors, and irrigation is performed by the irrigation device 120. Furthermore, the irrigation device 120 starts irrigation at a timing when a water content less than a preset irrigation threshold value is measured. Furthermore, the moisture sensor 300 starts measurement at regular intervals, and the measurement frequency thereof is lower than a predetermined value. However, within a period in which the water content satisfies the following expression, the moisture sensor 300 performs additional measurement in which the measurement frequency is higher than the predetermined value.

### Measurement Threshold Value)>(Water Content)>(Irrigation Threshold Value) . . . Expression 1

In the above expression, the measurement threshold value is a value higher than the irrigation threshold value, and is set, for example, by the user.

Furthermore, in line number LN2, the irrigation device 120 also decides a next irrigation amount in accordance with a measurement result of the water content. As described above, the measurement result of the water content is also used for control of the irrigation timing.

In line number LN3, an installation place (a cultivation place) of the moisture sensor 300 and the like is outdoors, and irrigation is performed by the irrigation device 120. Furthermore, since the installation place is outdoors, rainfall is also assumed. Furthermore, the irrigation device 120 performs irrigation when an irrigation time set by the user has elapsed. Furthermore, the moisture sensor 300 performs measurement of a water content at regular intervals only within the measurement period immediately before the irrigation time. The irrigation device 120 decides a next irrigation amount in accordance with the measurement result.

In line number LN4 as well, an installation place (a cultivation place) of the moisture sensor 300 and the like is outdoors, and irrigation is performed by the irrigation device 120. Furthermore, since the installation place is outdoors, rainfall is also assumed. Furthermore, the irrigation device 120 starts irrigation at a timing when a water content less than a preset irrigation threshold value is measured. Furthermore, the moisture sensor 300 starts measurement at regular intervals, and the measurement frequency thereof is lower than a predetermined value. However, within a period in which the water content satisfies Expression 1, the moisture sensor 300 performs additional measurement in which the measurement frequency is higher than the predetermined value. Furthermore, the irrigation device 120 decides a next irrigation amount in accordance with the measurement result of the water content. As described above, the measurement result of the water content is also used for control of the irrigation timing.

In line number LN5 as well, an installation place (a cultivation place) of the moisture sensor 300 and the like is outdoors. However, irrigation is not performed, and only rainfall is assumed. A measurement timing by the moisture sensor 300 is not fixed, and is performed, for example, before and after rainfall or at regular intervals.

Any one of the above five cultivation methods is selected by the user. In a case where line number LN1 or LN3 is selected, the user sets at least an irrigation time. Predetermined initial values are set in advance for the measurement interval and the measurement time of the water content, but the user can change those values.

Furthermore, in a case where line number LN2 or LN4 is selected, the user sets at least the measurement threshold value. Predetermined initial values are set in advance for the measurement interval and the measurement time of the water content of each of a case of high-frequency measurement and a case of low-frequency measurement, but the user can change the measurement interval and the measurement time of the high-frequency measurement.

As illustrated in b in FIG. 4, setting information corresponding to other than line number LN5 includes at least an irrigation time or a measurement threshold value. For example, setting information of identification information A corresponding to line number LN1 includes information indicating T_{A} as the irrigation time. Furthermore, setting information of identification information B corresponding to line number LN2 includes information indicating TH_{B}% as the measurement threshold value. Setting information of identification information C corresponding to line number LN3 includes information indicating T_{C} as the irrigation time. Furthermore, setting information of identification information D corresponding to line number LN4 includes information indicating TH_{D}% as the measurement threshold value. Furthermore, in a case where the user changes the measurement interval and the measurement time, the setting information further includes information indicating the changed values.

As illustrated in a and b in FIG. 4, the user can set a desired measurement method in accordance with a cultivation method by the user terminal 210 generating setting information in accordance with the cultivation method.

### Configuration Example of Moisture Sensor

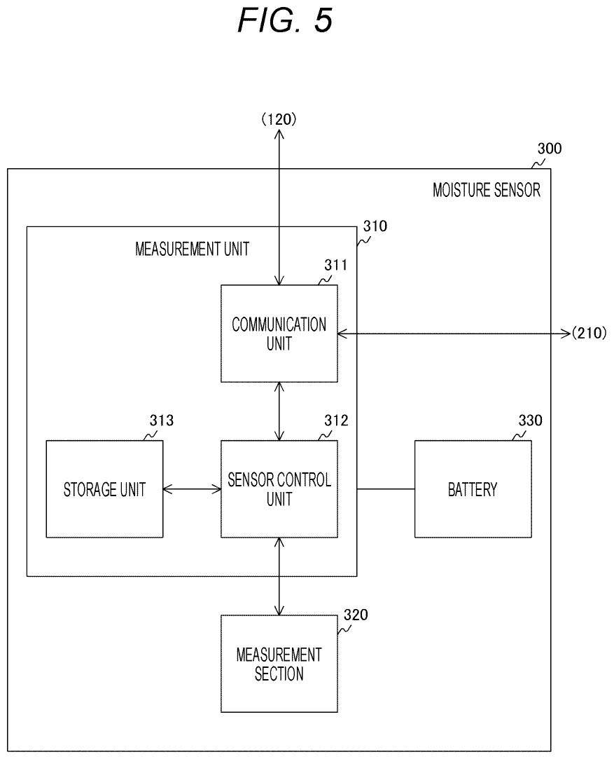

FIG. 5 is a block diagram illustrating a configuration example of the moisture sensor 300 in the first embodiment of the present technology. The moisture sensor 300 includes a measurement unit 310, a measurement section 320, and a battery 330. The measurement unit 310 includes a communication unit 311, a sensor control unit 312, and a storage unit 313.

The communication unit 311 transmits and receives various kinds of information to and from devices and equipment such as the user terminal 210 and the irrigation device 120. The communication unit 311 receives setting information from the user terminal 210. Furthermore, the communication unit 311 transmits measurement information to the irrigation device 120 and the user terminal 210.

The sensor control unit 312 controls the entire moisture sensor 300. The sensor control unit 312 controls the measurement section 320 on the basis of the setting information to measure the water content.

In a case where line number LN1 or LN3 is selected, an irrigation time is set by the user as described above. In this case, the sensor control unit 312 causes the water content to be measured at regular intervals only within a measurement period immediately before the irrigation time.

Furthermore, in a case where line number LN2 or LN4 is selected, a measurement threshold value is set by the user as described above. In this case, the sensor control unit 312 causes measurement to be started at a low frequency, and causes measurement to be performed at a high frequency within a period in which Expression 1 is satisfied.

Furthermore, the sensor control unit 312 generates measurement information indicating the measurement result of the measurement section 320, and transmits the measurement information to the irrigation device 120 and the user terminal 210 via the communication unit 311. Furthermore, the sensor control unit 312 obtains a remaining battery level of the battery 330 as necessary, and transmits information indicating the remaining battery level to the user terminal 210. Note that the sensor control unit 312 may transmit information (a terminal voltage or the like) necessary for calculation of the remaining battery level, and the remaining battery level may be calculated on the user terminal 210 side.

The storage unit 313 stores setting information and measurement information. The measurement section 320 is for measuring a water content of soil. The battery 330 supplies power to the measurement unit 310.

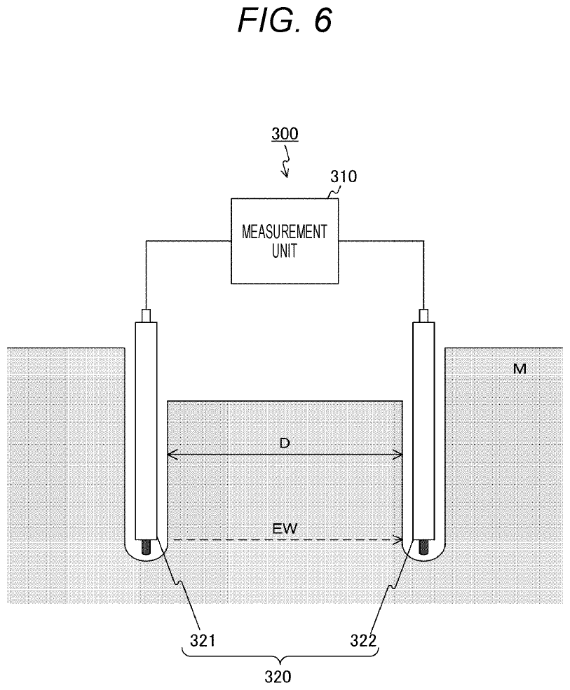

FIG. 6 is a diagram for describing a measurement method of the moisture sensor 300 in the first embodiment of the present technology. As the moisture sensor 300, for example, a sensor of Spatial Frequency Domain Transmissometry (SFDT) is used. In the SFDT method, the measurement section 320 includes a transmission probe 321 and a reception probe 322. These probes are embedded in soil M at a predetermined interval D.

The measurement unit 310 causes an electromagnetic wave EW to be transmitted to the transmission probe 321 and causes the electromagnetic wave to be received by the reception probe 322. A frequency of the electromagnetic wave EW is swept within a predetermined frequency range. For example, the frequency is swept within a range from 1 to 9 gigahertz (GHz) at intervals of 50 megahertz (MHz). In this case, the measurement unit 310 performs detection at 161 points within a frequency range, and an S-parameter is obtained by a vector network analyzer or the like. The measurement unit 310 calculates a water content by calculation for the S-parameter.

Furthermore, in a case where it is desired to improve a Signal-Noise (SN) ratio, the measurement unit 310 sweeps the frequency multiple times within the frequency range, calculates the S-parameter multiple times, and calculates the water content from an average value thereof.

Note that a sensor of a method other than the SFDT method, such as a Time Domain Reflectometry (TDR) method or a capacitance method, can also be used as the moisture sensor 300.

### Operation Example of User Terminal

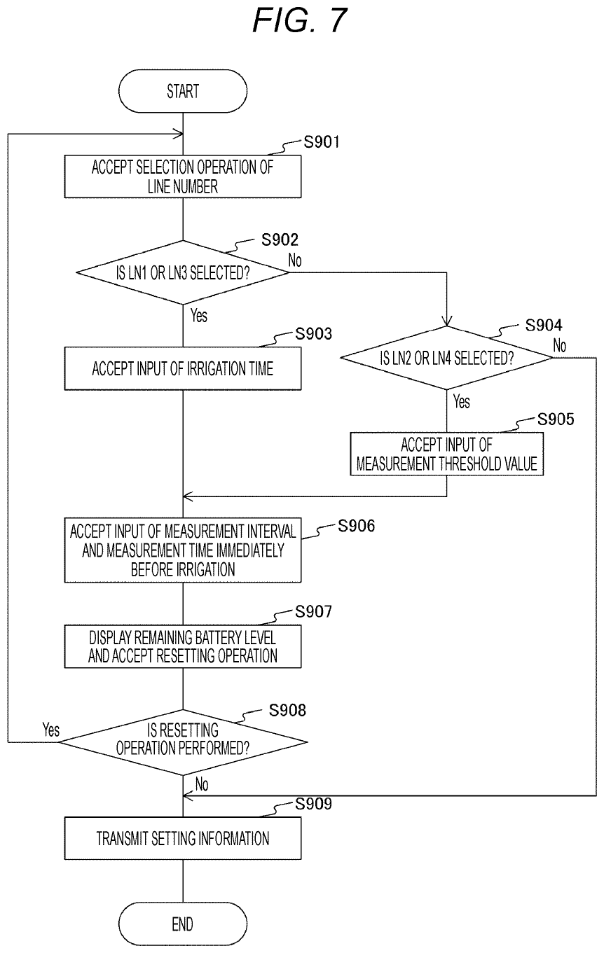

FIG. 7 is a flowchart illustrating an example of an operation of the user terminal 210 in the first embodiment of the present technology. This operation is started when, for example, a predetermined application for performing settings is executed.

The user terminal 210 accepts a selection operation of a line number and holds the selected line number (step S901). The user terminal 210 determines whether or not any of line number LN1 or LN3 is selected (step S902).

In a case where any of line number LN1 or LN3 is selected (step S902: Yes), the user terminal 210 accepts an input of an irrigation time and holds the input irrigation time (step S903).

On the other hand, in a case where any of line number LN2, LN4, or LN5 is selected (step S902: No), the user terminal 210 determines whether or not any of line number LN2 or LN4 is selected (step S904). In a case where any of line number LN2 or LN4 is selected (step S904: Yes), the user terminal 210 accepts an input of a measurement threshold value and holds the input measurement threshold value (step S905).

After step S903 or S905, the user terminal 210 accepts an input of a measurement interval and a measurement time immediately before irrigation, and holds the input values (step S906). Note that step S906 is executed in a case where the user changes at least one initial value of the measurement interval or the measurement time.

Then, the user terminal 210 displays the remaining battery level and accepts a resetting operation by the user (step S907). The user terminal 210 determines whether or not the resetting operation has been performed (step S908). In a case where the resetting operation has been performed (step S908: Yes), the user terminal 210 repeatedly executes step S901 and subsequent steps.

On the other hand, in a case where the resetting operation has not been performed (step S908: No) or in a case where line number LN5 is selected (step S904: No), the user terminal 210 generates and transmits setting information (step S909). After step S909, the user terminal 210 ends an operation for settings.

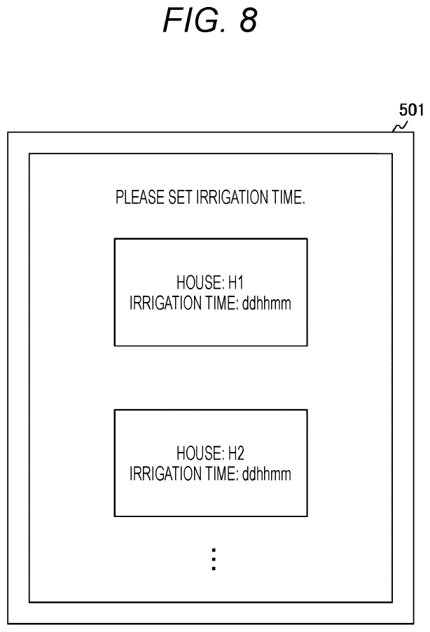

Subsequently, with reference to FIGS. 8 to 11, setting screens to be displayed on the user terminal 210 will be described.

FIG. 8 is an example of a setting screen 501 for irrigation time in the first embodiment of the present technology. When line number LN1 or LN3 is selected, the user terminal 210 displays the setting screen of FIG. 8. For example, in a case Where there are two or more cultivation places, the user can set an irrigation time for each place. In FIG. 8, the irrigation time is set for each of houses H1 and H2.

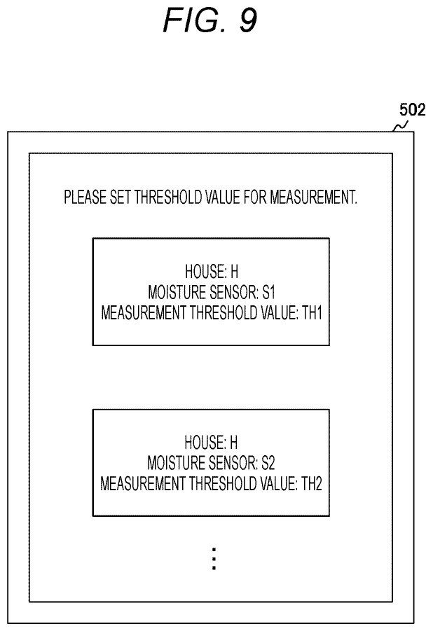

FIG. 9 is an example of a setting screen 502 for a threshold value of a water content in the first embodiment of the present technology. When line number LN2 or LN4 is selected, the user terminal 210 displays the setting screen of FIG. 9. For example, in a case where two or more moisture sensors 300 are installed, the user can set a measurement threshold value for each sensor. In FIG. 9, a measurement threshold value is set for each of moisture sensors S1 and S2.

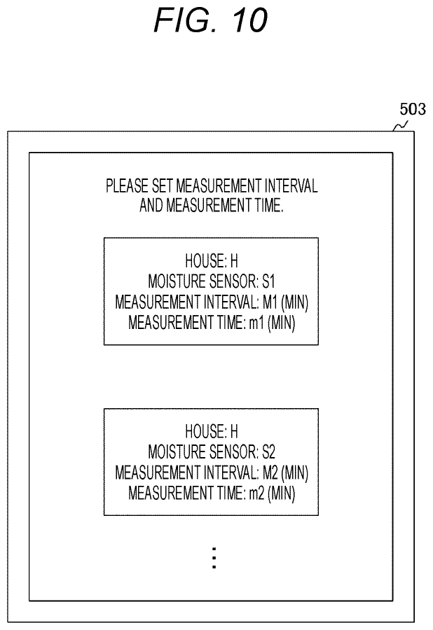

FIG. 10 is an example of a setting screen 503 for a measurement interval and a measurement time in the first embodiment of the present technology. When the user performs an operation for changing values of the measurement interval and the measurement time, the user terminal 210 displays the setting screen of FIG. 10. For example, in a case where two or more moisture sensors 300 are installed, the user can set a measurement interval and a measurement time for each sensor. In FIG. 10, the measurement interval and the measurement time are set for each of the moisture sensors S1 and S2.

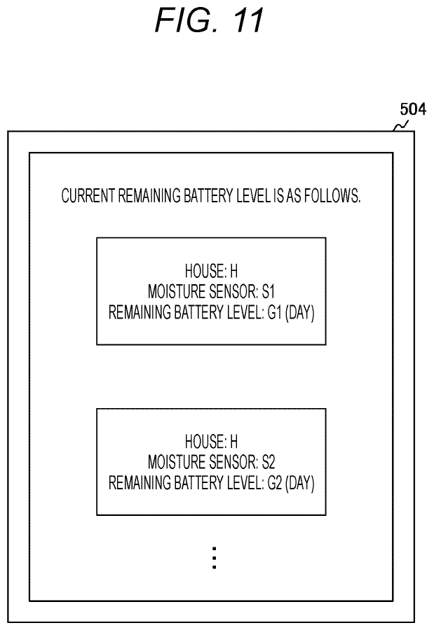

FIG. 11 is an example of a display screen 504 for a remaining battery level in the first embodiment of the present technology. After the user performs the settings illustrated in FIGS. 8 to 10, the user terminal 210 displays a screen of FIG. 11 as necessary. For example, in a case where two or more moisture sensors 300 are installed, a remaining battery level is displayed for each sensor. The remaining battery level can also be expressed in milliampere hours (mAh), but in FIG. 11, from a viewpoint of user convenience, the remaining battery level is expressed by conversion into the number of days for which the battery lasts. This number of days varies in accordance with various setting contents illustrated in FIGS. 8 to 10. The user checks the remaining battery level and performs resetting when necessary.

Note that the user terminal 210 can propose setting contents for further power saving by an application. In this case, for example, a message such as “When the number of measurements or the measurement time is changed to XX, the number of days for which the battery lasts is improved to YY days” is displayed on the user terminal 210.

Furthermore, the user terminal 210 separately provides the setting screen of FIG. 10 and the display screen of the remaining battery level of FIG. 11, but can also collectively display the display contents of these screens on one screen.

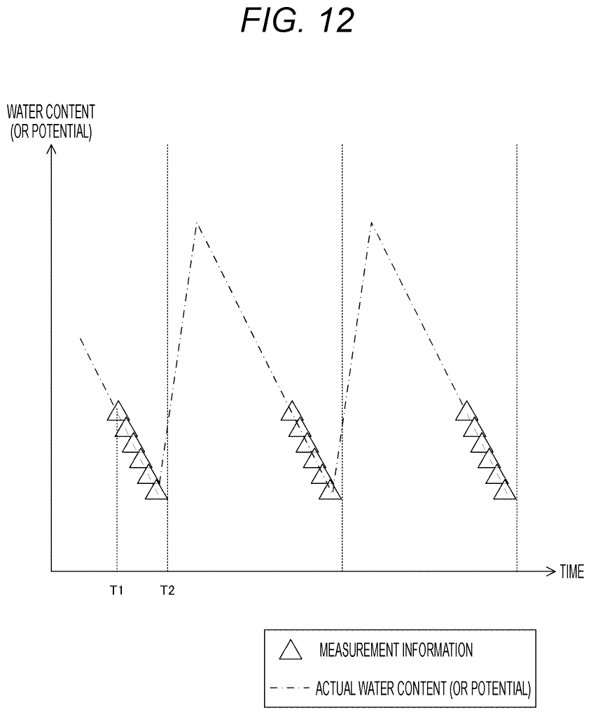

Next, with reference to FIGS. 12 to 15, measurement methods of line numbers LN1 to L4 will be described.

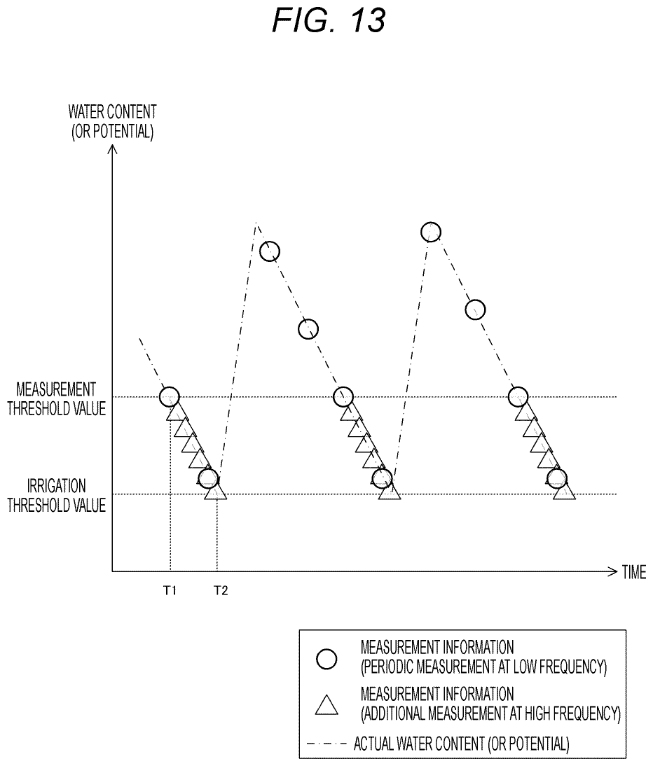

FIG. 12 is a diagram for describing a measurement method in a case of line number LN1 supplied with water indoors in the first embodiment of the present technology. In the drawing, a vertical axis indicates a water content, and a horizontal axis indicates time. The same applies to FIGS. 13 to 15. Note that in a case where the water potential sensor 400 is used, a water potential is measured instead of the water content.

In FIG. 12, a triangle indicates measurement information, and a one-dot chain line indicates an actual water content. When an irrigation time such as time T2 has elapsed, the water content increases. The moisture sensor 300 measures a water content at regular intervals within a measurement period from time T1, which is a predetermined period before the irrigation time, to time T2. Note that the measurement period from time T1 to time T2 in FIG. 12 is an example of a first measurement period described in the claims.

Furthermore, in FIG. 12, the moisture sensor 300 performs measurement only within a measurement period immediately before irrigation, and does not perform measurement outside the measurement period, but is not limited to this control. The moisture sensor 300 can also perform measurement even in a period not corresponding to the measurement period in FIG. 12 (such as before time T1), and can make the measurement frequency lower than the measurement frequency within the measurement period immediately before irrigation.

As illustrated in FIG. 12, the measurement frequency of the water content within the measurement period (such as a period from time T1 to time T2) immediately before irrigation is higher than a predetermined value. On the other hand, outside the measurement period, the measurement frequency is lower than the predetermined value.

FIG. 13 is a diagram for describing a measurement method in a case of line number LN2 supplied with water indoors in the first embodiment of the present technology. In the drawing, a circle mark indicates measurement information of periodic low-frequency measurement, and a triangle indicates measurement information of additional measurement performed at a high frequency. A one-dot chain line indicates an actual water content. For example, a measurement interval of the circle mark is set to 60 minutes, and a measurement interval of the triangle is set to 10 minutes.

The moisture sensor 300 starts periodic measurement at a low frequency. When a water content less than a measurement threshold value is measured at time T1 or the like, the moisture sensor 300 starts additional measurement at a high frequency. Then, when a water content less than an irrigation threshold value is measured at time T2 or the like, irrigation is executed and the water content increases. After this time T2 has elapsed, the moisture sensor 300 ends additional measurement at a high frequency. In this manner, within a period from time T1 immediately before irrigation to time T2, the moisture sensor 300 performs additional measurement at a high frequency while continuing periodic low-frequency measurement. Therefore, the measurement frequency of the water content within the measurement period immediately before irrigation is higher than a predetermined value, while the measurement frequency is lower than the predetermined value outside the measurement period. Note that the moisture sensor 300 can switch the measurement frequency from a low frequency to a high frequency in the measurement period immediately before irrigation, without performing additional measurement.

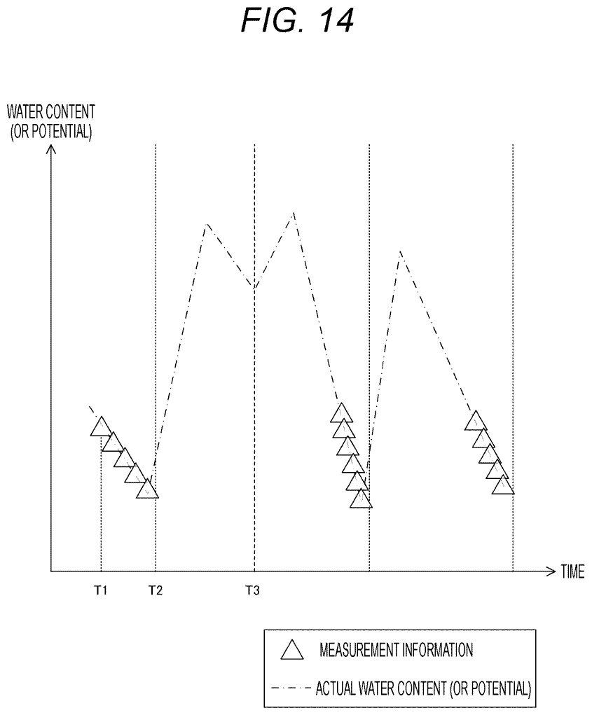

FIG. 14 is a diagram for describing a measurement method in a case of line number LN3 supplied with water outdoors in the first embodiment of the present technology. In the drawing, a triangle indicates measurement information, and a one-dot chain line indicates an actual water content. When an irrigation time such as time T2 has elapsed, the water content increases. Furthermore, when a rainfall time such as time T3 has elapsed, the water content increases. The measurement method in the drawing is similar to that of line number LN1.

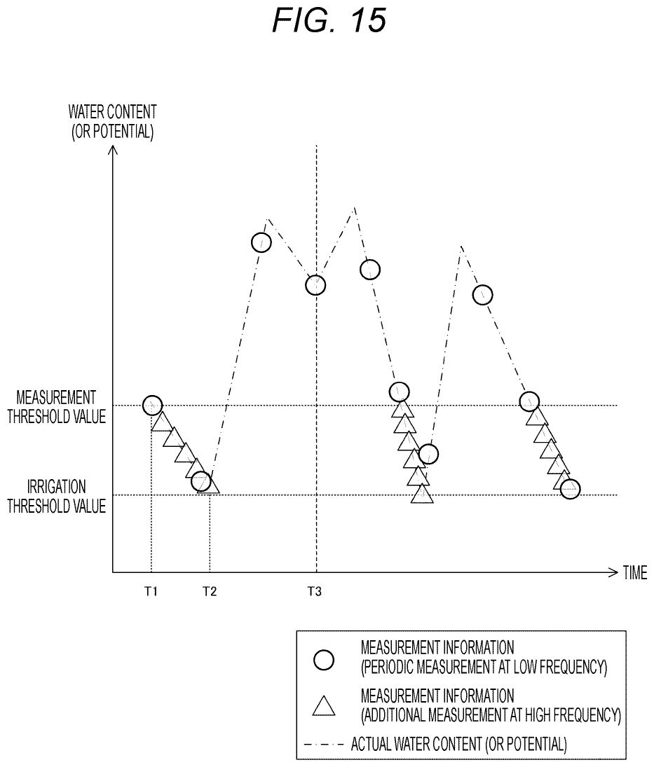

FIG. 15 is a diagram for describing a measurement method in a case of line number LN4 supplied with water outdoors in the first embodiment of the present technology. In the drawing, a circle mark indicates measurement information of periodic low-frequency measurement, and a triangle indicates measurement information of additional measurement performed at a high frequency. A one-dot chain line indicates an actual water content. When an irrigation time such as time T2 has elapsed, the water content increases. Furthermore, when a rainfall time such as time T3 has elapsed, the water content increases. The measurement method in the drawing is similar to that of line number LN2.

As illustrated in FIGS. 12 to 15, the moisture sensor 300 controls the measurement frequency of the water content within the measurement period immediately before irrigation to a value higher than a predetermined value, and controls the measurement frequency outside the measurement period to a value lower than the predetermined value.

In actual cultivation, it is preferable to increase the measurement frequency immediately before irrigation in order to accurately measure the water content. However, since the water content other than that is not used as an index for irrigation, there is a case where there is no problem in practical use even when the measurement frequency is low. Therefore, by the moisture sensor 300 performing measurement at a high frequency within the measurement period immediately before irrigation, it is possible to reduce the power consumption of the moisture sensor 300 as compared with a case where the measurement is always performed at a high frequency, while ensuring practicality. Therefore, a duration of the battery 330 can be extended.

Note that in FIGS. 12 to 15, the user terminal 210 acquires an irrigation timing from the irrigation time or the irrigation threshold value in the setting information, but is not limited to this configuration. For example, the irrigation device 120 can generate a control signal instructing a start and an end of the measurement period on the basis of the setting information, and transmit the control signal to the user terminal 210. In this case, the user terminal 210 does not need to acquire an irrigation timing from the setting information.

As described above, according to the first embodiment of the present technology, the moisture sensor 300 makes the measurement frequency of the water content within the measurement period immediately before irrigation higher than the predetermined value, and makes the measurement frequency outside the measurement period lower than the predetermined value, and therefore, it is possible to reduce the power consumption.

### Modified Example

In the first embodiment described above, the moisture sensor 300 controls the measurement frequency, but is not limited to this, and a device external to the moisture sensor 300 can also control the measurement frequency. The measurement system 200 in a modified example of the first embodiment is different from that of the first embodiment in that a control device external to the moisture sensor 300 controls the measurement frequency.

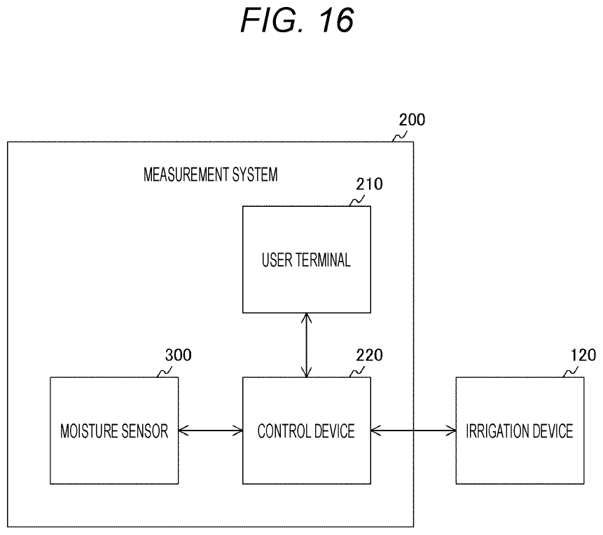

FIG. 16 is a block diagram illustrating a configuration example of the measurement system 200 in the modified example of the first embodiment of the present technology. The measurement system 200 in the modified example of the first embodiment is different from that of the first embodiment in that the measurement system 200 further includes a control device 220 in addition to the user terminal 210 and the moisture sensor 300. The user terminal 210 generates setting information and transmits the setting information to the control device 220.

The control device 220 integrally controls the moisture sensor 300 and the irrigation device 120. A data logger or the like is used as the control device 220.

Note that the number of moisture sensors 300 in the measurement system 200 is optional, and two or more moisture sensors 300 may be disposed. The number of irrigation devices 120 is also optional, and two or more irrigation devices 120 may be disposed.

Furthermore, the user performs settings using the user terminal 210, but the present technology is not limited to this configuration. The user may input setting information directly to the control device 220 without using the user terminal 210 when performing the settings.

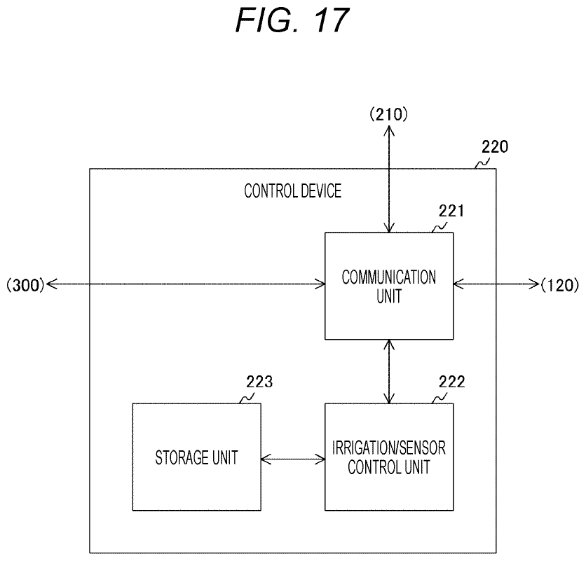

FIG. 17 is a block diagram illustrating a configuration example of the control device 220 in the modified example of the first embodiment of the present technology. The control device 220 includes a communication unit 221, an irrigation/sensor control unit 222, and a storage unit 223.

The communication unit 221 transmits and receives various kinds of information between the user terminal 210, the moisture sensor 300, and the irrigation device 120.

The irrigation/sensor control unit 222 receives setting information from the user terminal 210 via the communication unit 221. Then, on the basis of the setting information, the irrigation/sensor control unit 222 generates an irrigation control signal for controlling the irrigation device 120, and causes the irrigation device 120 to perform irrigation via the communication unit 221. Furthermore, on the basis of the setting information, the irrigation/sensor control unit 222 generates a sensor control signal for controlling the moisture sensor 300, transmits the sensor control signal to the moisture sensor 300 via the communication unit 221, and causes the water content to be measured. A control method of the irrigation timing of the irrigation device 120 and the measurement frequency of the moisture sensor 300 is similar to that in the first embodiment. Furthermore, the irrigation/sensor control unit 222 receives measurement information from the moisture sensor 300 via the communication unit 221, and uses the measurement information for control of the irrigation device 120. The storage unit 223 stores setting information and measurement information.

As illustrated in FIGS. 16 and 17, instead of the moisture sensor 300, the control device 220 controls a measurement frequency of a water content within a measurement period immediately before irrigation to a value higher than a predetermined value, and controls the measurement frequency outside the measurement period to a value lower than the predetermined value by means of a sensor control signal. Therefore, a processing load of the moisture sensor 300 can be reduced.

As described above, according to the modified example of the first embodiment of the present technology, since the control device 220 controls the measurement frequency, the processing load of the moisture sensor 300 can be reduced.

### 2. Second Embodiment

In the first embodiment described above, the moisture sensor 300 increases the measurement frequency immediately before irrigation. However, in order to check whether or not irrigation has been appropriately performed, there is a case where high-frequency measurement is required immediately after irrigation as well. The moisture sensor 300 in a second embodiment is different from that of the first embodiment in that the measurement frequency is increased immediately before irrigation and immediately after irrigation.

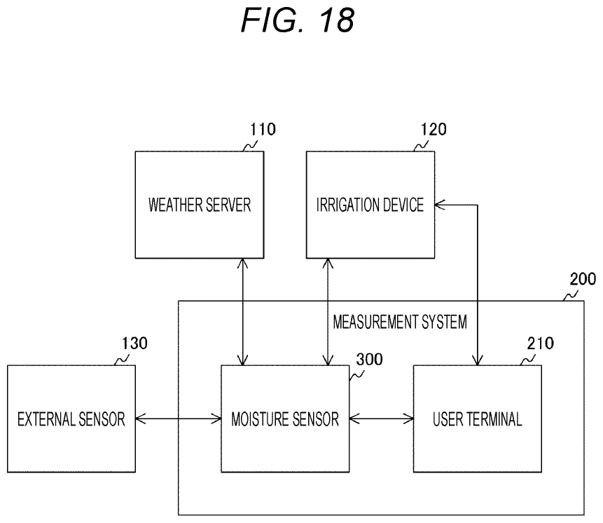

FIG. 18 is a block diagram illustrating a configuration example of the measurement system 200 in the second embodiment of the present technology. The moisture sensor 300 in the second embodiment can receive rainfall information and the like from a weather server 110 and use the rainfall information and the like for control.

Furthermore, the moisture sensor 300 can access an external sensor 130. As the external sensor 130, for example, another moisture sensor, a camera, or a Global Positioning System (GPS) sensor is used. The moisture sensor 300 can receive image data or position information from the camera or the GPS sensor and use the image data or position information for control.

Furthermore, similarly to the first embodiment, in the second embodiment, a user can change the measurement interval and the measurement time of the water content “immediately before irrigation”.

Furthermore, in the second embodiment, the water content is also measured within the measurement period “immediately after irrigation”. Predetermined initial values are set in advance for these measurement intervals and the measurement times, but the user can change those values.

Furthermore, in line numbers LN3 and LN4 supplied with water outdoors, the moisture sensor 300 can perform measurement at a high frequency not only immediately after irrigation but also immediately after rainfall. The measurement interval and the measurement time immediately after rainfall are set, for example, to the same values as those immediately after irrigation.

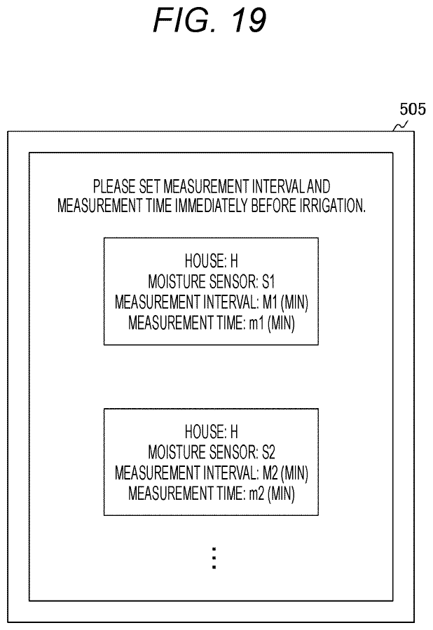

As illustrated in FIG. 19, when an operation for changing the measurement interval and the measurement time “immediately before irrigation” is performed, the user terminal 210 displays a setting screen 505 for the operation.

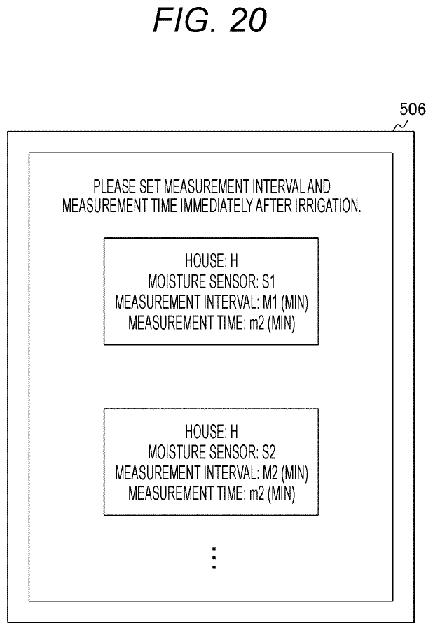

Furthermore, as illustrated in FIG. 20, when an operation for changing the measurement interval and the measurement time “immediately after irrigation” is performed, the user terminal 210 displays a setting screen 506 for the operation.

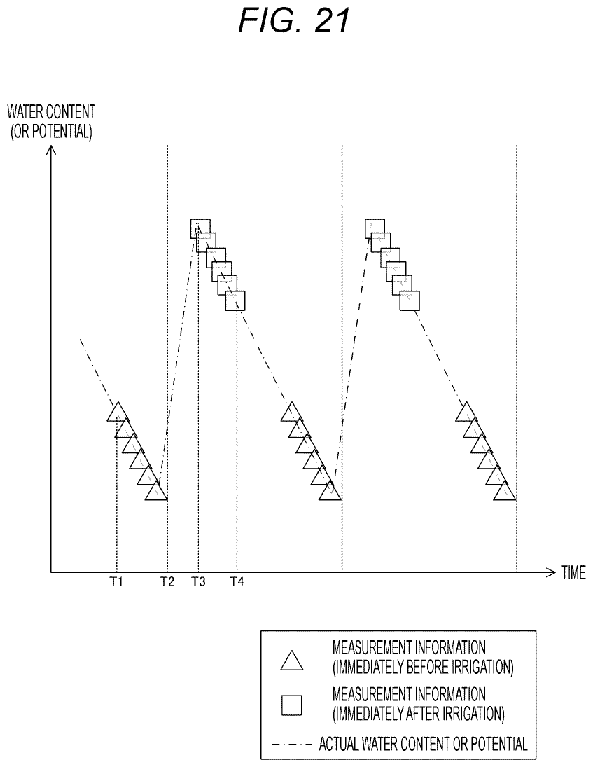

Next, with reference to FIGS. 21 to 24, measurement methods of line numbers LN1 to L4 will be described.

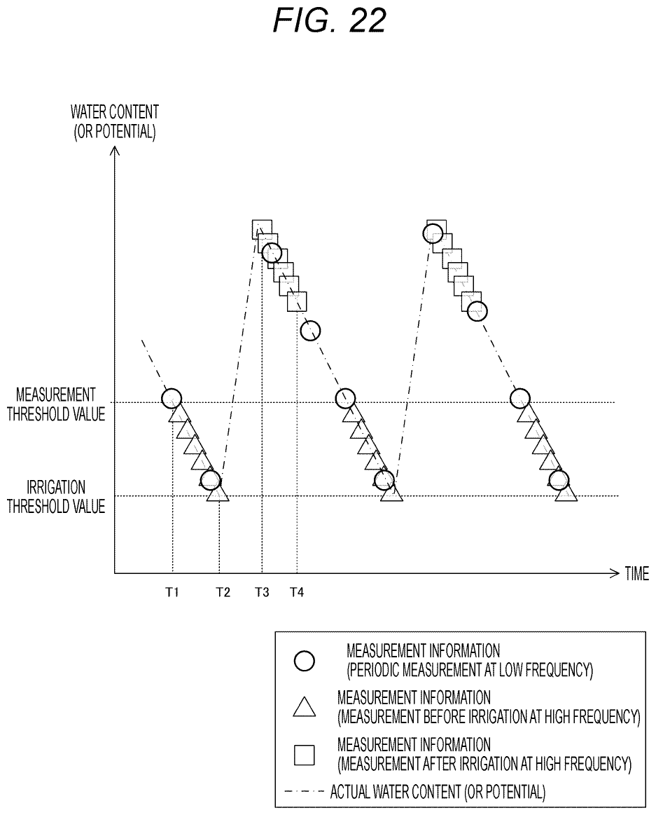

FIG. 21 is a diagram for describing a measurement method in a case of line number LN1 supplied with water indoors in the second embodiment of the present technology. In the drawing, a vertical axis indicates a water content, and a horizontal axis indicates time. The same applies to FIGS. 22 to 24. Note that in a case where the water potential sensor 400 is used, a water potential is measured instead of the water content.

In FIG. 21, a triangle indicates measurement information immediately before irrigation, and a one-dot chain line indicates an actual water content. A square indicates measurement information immediately after irrigation. The moisture sensor 300 measures a water content at regular intervals within a measurement period from time T1, which is a predetermined period before T2 as the irrigation time, to time T2. Furthermore, a time immediately after irrigation is defined as T3, and a time after a predetermined time has elapsed from T3 is defined as T4. The moisture sensor 300 measures a water content at regular intervals also within a measurement period from time T3 immediately after irrigation to time T4. Note that the measurement period from time T3 to time T4 is an example of a second measurement period described in the claims.

As illustrated in FIG. 21, in the measurement period (such as period from time T1 to time T2) immediately before irrigation and the measurement period (such as period from time T3 to time T4) immediately after irrigation, the measurement frequency of the water content is higher than a predetermined value. On the other hand, outside these measurement periods, the measurement frequency is lower than the predetermined value.

FIG. 22 is a diagram for describing a measurement method in a case of line number LN2 supplied with water indoors in the second embodiment of the present technology. In the drawing, a circle mark indicates measurement information of periodic low-frequency measurement, and a triangle indicates measurement information of additional measurement performed immediately before irrigation at a high frequency. A square indicates measurement information of additional measurement performed immediately after irrigation at a high frequency. A one-dot chain line indicates an actual water content.

The moisture sensor 300 starts periodic measurement at a low frequency. When a water content less than a measurement threshold value is measured at time T1 or the like, the moisture sensor 300 starts additional measurement at a high frequency. Then, when a water content less than an irrigation threshold value is measured at time T2 or the like, the moisture sensor 300 ends additional measurement at a high frequency. Furthermore, a time immediately after irrigation is defined as T3, and a time after a predetermined time has elapsed from T3 is defined as T4. The moisture sensor 300 performs additional measurement at a high frequency also within a period from time T3 immediately after irrigation to time T4. Note that the moisture sensor 300 can also continuously perform additional measurement at a high frequency throughout a period from time T1 to time T4.

In this manner, within the measurement period from time T1 immediately before irrigation to time T2, the moisture sensor 300 performs additional measurement at a high frequency while continuing periodic low-frequency measurement. Furthermore, within the measurement period from time T3 immediately after irrigation to time T4, the moisture sensor 300 performs additional measurement at a high frequency while continuing periodic low-frequency measurement. Therefore, in the measurement period immediately before irrigation and the measurement period immediately after irrigation, the measurement frequency of the water content is higher than a predetermined value, and outside these periods, the measurement frequency is lower than the predetermined value. Note that the moisture sensor 300 can switch the measurement frequency from a low frequency to a high frequency in the measurement period immediately before and immediately after irrigation, without performing additional measurement.

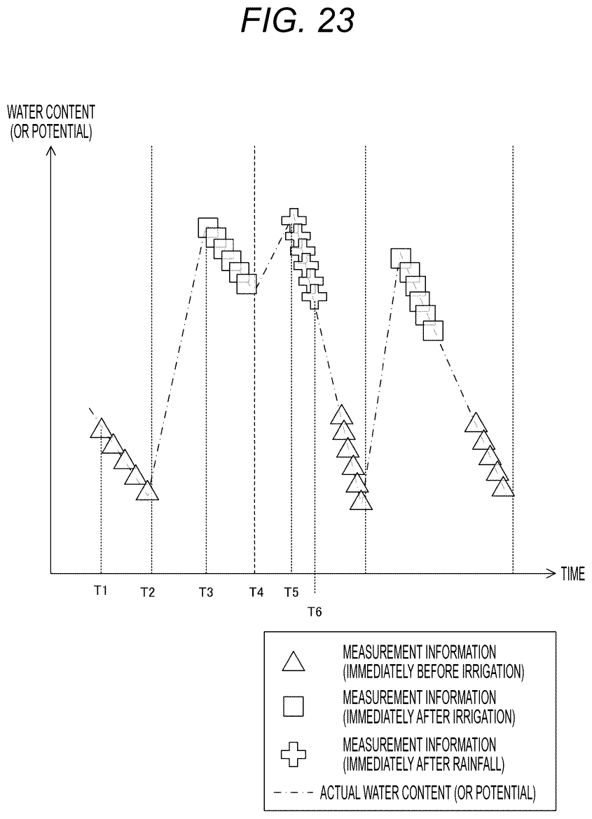

FIG. 23 is a diagram for describing a measurement method in a case of line number LN3 supplied with water outdoors in the second embodiment of the present technology. A square indicates measurement information immediately after irrigation, and a cross mark indicates measurement information immediately after rainfall. In the drawing, a triangle indicates measurement information, and a one-dot chain line indicates an actual water content. The measurement method immediately before irrigation and immediately after irrigation is similar to that of line number LN1.

Furthermore, the moisture sensor 300 acquires, for example, rainfall information transmitted from the weather server 110, and rainfall time from the external sensor 130 (such as another moisture sensor). A time immediately after rainfall is defined as T5, and a time after a predetermined time has elapsed from T5 is defined as T6. The moisture sensor 300 measures a water content at regular intervals also within a measurement period from time T5 immediately after rainfall to time T6. Note that the measurement period from time T5 to time T6 is an example of a third measurement period described in the claims.

As illustrated in FIG. 23, in the measurement period immediately before irrigation and immediately after irrigation and the measurement period (such as a period from time T5 to time T6) immediately after rainfall, the measurement frequency of the water content is higher than the predetermined value. On the other hand, outside these measurement periods, the measurement frequency is lower than the predetermined value.

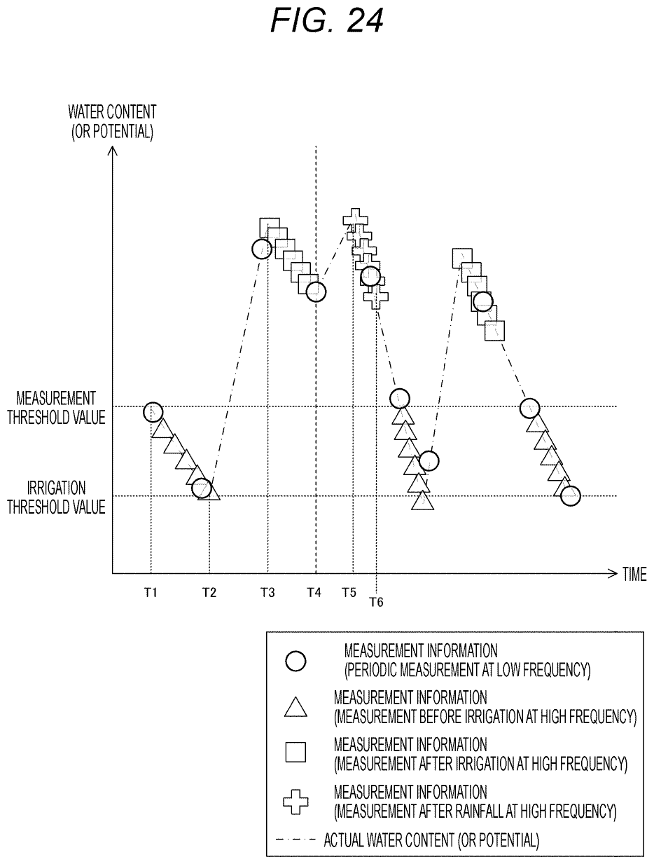

FIG. 24 is a diagram for describing a measurement method in a case of line number LN4 supplied with water outdoors in the second embodiment of the present technology. In the drawing, a circle mark indicates measurement information of periodic low-frequency measurement, and a triangle indicates measurement information of additional measurement performed immediately before irrigation at a high frequency. A square indicates measurement information of additional measurement performed immediately after irrigation at a high frequency, and a cross mark indicates measurement information of additional measurement performed immediately after rainfall at a high frequency. A one-dot chain line indicates an actual water content. The low-frequency measurement method and the high-frequency measurement method immediately before irrigation and immediately after irrigation are similar to those of line number LN2.

Within the period from time T5 immediately after rainfall to time T6, the moisture sensor 300 performs additional measurement at a high frequency while continuing periodic low-frequency measurement. Alternatively, the moisture sensor 300 switches the measurement frequency from a low frequency to a high frequency within the period. Therefore, in the measurement period immediately before irrigation and immediately after irrigation and the measurement period immediately after rainfall, the measurement frequency of the water content is higher than a predetermined value, and outside these periods, the measurement frequency is lower than the predetermined value.

As described above, by increasing the measurement frequency immediately after irrigation, the user can check whether or not irrigation has been appropriately performed. As a result, user convenience can be improved. Furthermore, by increasing the measurement frequency immediately after rainfall, the user can check the influence of rainfall, and the irrigation device 120 can perform irrigation control in consideration of the influence of rainfall. Furthermore, the measurement system 200 can accurately predict a change in water content over time with a minimum number of measurements.

Note that the modified example of the first embodiment can be applied to the second embodiment.

As described above, according to the second embodiment of the present technology, since the moisture sensor 300 makes the measurement frequency immediately after irrigation in addition to immediately before irrigation higher than the predetermined value, user convenience can be improved.

### 3. Third Embodiment

In the first embodiment described above, the moisture sensor 300 controls the measurement frequency, but is not limited to this configuration. The moisture sensor 300 in a third embodiment is different from that of the first embodiment in that the moisture sensor 300 controls measurement accuracy instead of the measurement frequency.

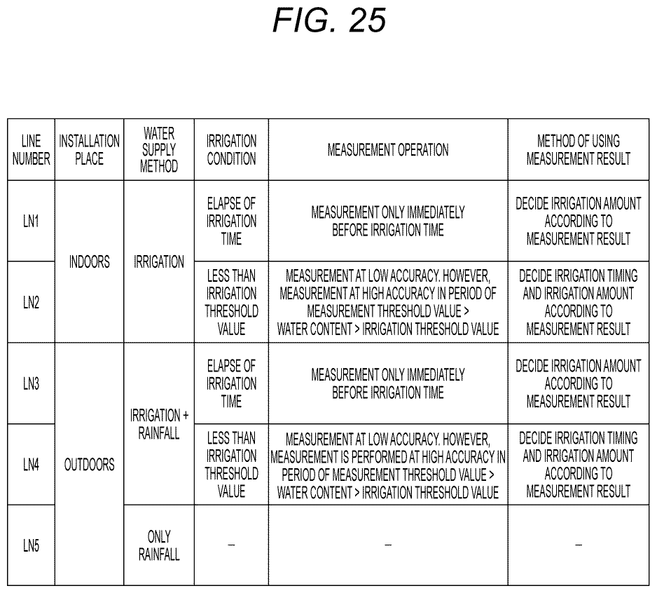

FIG. 25 is a diagram for describing setting contents in the third embodiment of the present technology. In a case where line numbers LN2 and LN4 are selected, the moisture sensor 300 performs periodic measurement at a low accuracy. However, within a period satisfying Expression 1, the moisture sensor 300 performs periodic measurement at a high accuracy.

For example, the moisture sensor 300 controls measurement accuracy by changing the number of sweep points of frequencies within a frequency range. In a case where the moisture sensor 300 performs frequency sweep at 50 megahertz (MHz) intervals within a range from 1 to 9 gigahertz (GHZ) during a high-accuracy period, for example, during a low-accuracy period, it is only required to reduce the number of sweep points by performing frequency sweep at 500 megahertz (MHz) intervals within the range.

Alternatively, the moisture sensor 300 controls measurement accuracy by changing a frequency range. In a case where the moisture sensor 300 performs frequency sweep within a range from 1 to 9 gigahertz (GHz) during a high-accuracy period, for example, during a low-accuracy period, it is only required to narrow the sweep range to a range from 1 to 6 gigahertz (GHz).

Alternatively, the moisture sensor 300 controls measurement accuracy by changing the number of times of averaging. In a case where the moisture sensor 300 measures S-parameters multiple times during a high-accuracy period and calculates a water content from an average value thereof, for example, during a low-accuracy period, it is only required to measure the S-parameter only once and reduce the number of times of averaging.

As described above, by the moisture sensor 300 controlling the measurement accuracy on the basis of setting information, it is possible to reduce power consumption of the moisture sensor 300 as compared with a case where measurement is always performed at a high accuracy.

Note that the modified example of the first embodiment and the second embodiment can be applied to the third embodiment.

As described above, according to the third embodiment of the present technology, since the moisture sensor 300 controls the measurement accuracy on the basis of setting information, it is possible to reduce power consumption.

### 4. Fourth Embodiment

In the first embodiment described above, the moisture sensor 300 measures the water content, but in a case where soil is relatively dry, the water potential sensor 400 can perform measurement with a wider dynamic range and a higher resolution. The measurement system 200 in a fourth embodiment is different from that of the first embodiment in that either the moisture sensor 300 or the water potential sensor 400 is switched according to a water content.

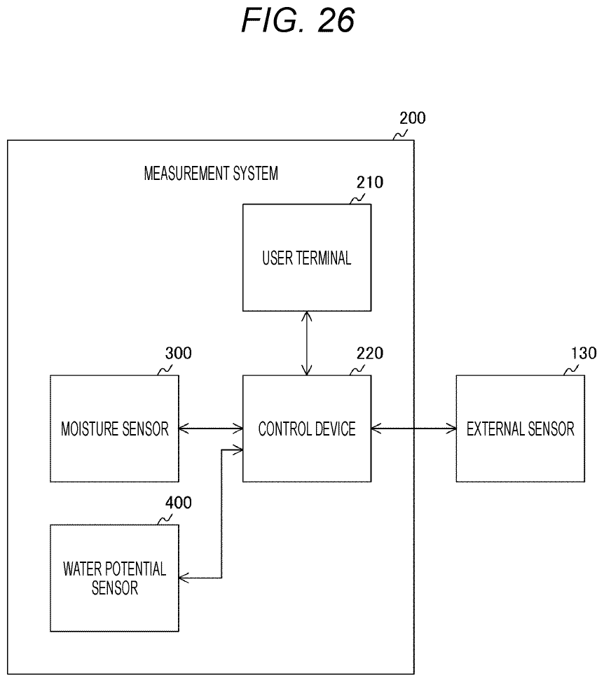

FIG. 26 is a block diagram illustrating a configuration example of the measurement system 200 in the fourth embodiment of the present technology. The measurement system 200 in the fourth embodiment is different from that of the first embodiment in that the measurement system 200 further includes the control device 220 and the water potential sensor 400 in addition to the user terminal 210 and the moisture sensor 300.

The control device 220 integrally controls the moisture sensor 300, the water potential sensor 400, and the irrigation device 120. Furthermore, the control device 220 performs control to switch between the moisture sensor 300 and the water potential sensor 400 according to the water content. Details of control contents will be described later.

Note that the number of moisture sensors 300 in the measurement system 200 is optional, and two or more moisture sensors 300 may be disposed. The number of irrigation devices 120 is also optional, and two or more irrigation devices 120 may be disposed.

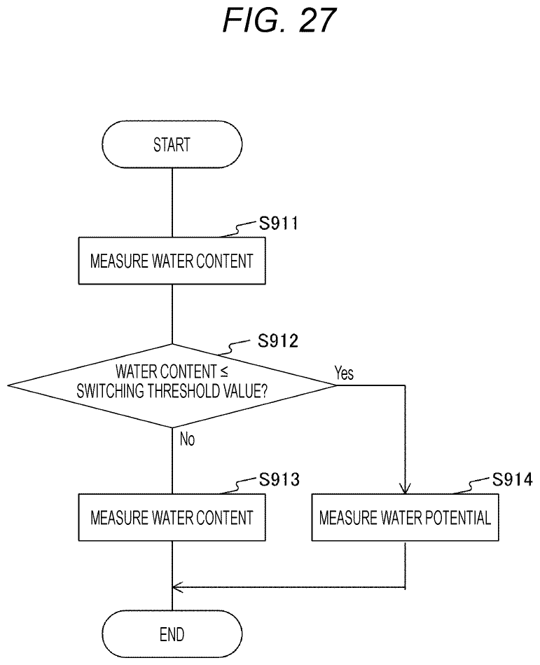

FIG. 27 is a flowchart illustrating an example of an operation of the control device 220 in the fourth embodiment of the present technology. This operation starts, for example, when a measurement start time immediately before irrigation or immediately after irrigation has elapsed.

The control device 220 controls the moisture sensor 300 to measure a water content while stopping a measurement operation of the water potential sensor 400 (step S911).

Then, it is determined whether or not the measured water content is equal to or lower than a predetermined switching threshold value (step S912). In a case where the water content exceeds the predetermined switching threshold value (step S912: No), the control device 220 continues to cause the moisture sensor 300 to measure the water content while stopping the measurement operation of the water potential sensor 400 (step S913).

On the other hand, in a case where the water content is equal to or lower than the predetermined switching threshold value (step S912: Yes), the control device 220 stops the measurement operation of the moisture sensor 300 and causes the water potential sensor 400 to measure a water potential (step S913). The measurement of step S913 or step S914 is continuously executed until the measurement period immediately before irrigation or immediately after irrigation ends.

Furthermore, the control illustrated in the drawing is executed for each of line numbers LN1 to LN4. As illustrated in the drawing, by performing the measurement by switching between the moisture sensor 300 and the water potential sensor 400 according to the water content, it is possible to perform measurement with an appropriate sensor and thereby improve convenience. Furthermore, by allowing only one of the moisture sensor 300 and the water potential sensor 400 to perform measurement, it is possible to reduce power consumption of the measurement system 200 as compared with a case where both of the moisture sensor 300 and the water potential sensor 400 perform measurement.

Note that the second embodiment and the third embodiment can be applied to the fourth embodiment.

As described above, according to the fourth embodiment of the present technology, since the control device 220 switches between the moisture sensor 300 and the water potential sensor 400, it is possible to improve convenience and to reduce power consumption.

Note that the embodiments described above indicate examples for embodying the present technology, and the respective matters in the embodiments and the respective matters specifying the invention in the claims have correspondence relationships. Similarly, the matters specifying the invention in the claims and the matters in the embodiments of the present technology denoted by the same names as the matters specifying the invention have a correspondence relationship. However, the present technology is not limited to the embodiments, and can be embodied by applying various modifications to the embodiments without departing from the scope of the present technology.

Furthermore, the procedures described in the above-described embodiments may be considered as a method including a series of procedures, and may be considered as a program for allowing a computer to execute the series of procedures or a recording medium that stores the program. As this recording medium, for example, a Compact Disc (CD), a MiniDisc (MD), a Digital Versatile Disc (DVD), a memory card, a Blu-ray (registered trademark) disc, and the like can be used.

Note that the effects described in the present specification are merely illustrative and are not limited, and furthermore, other effects may be provided.

Note that the present technology can also have the following configurations.

(1) A sensor including:
a measurement section that measures a parameter related to moisture of soil; and
a control unit that controls a measurement frequency of the parameter within a first measurement period immediately before an irrigation timing to the soil to a value higher than a predetermined value, and controls the measurement frequency before the first measurement period to a value lower than the predetermined value.

(2) The sensor according to (1), further including:
a communication unit that receives predetermined setting information.

(3) The sensor according to (2),
in which the setting information includes the irrigation timing, and
the first measurement period is a period from a timing, which is a predetermined time before the irrigation timing, to the irrigation timing.

(4) The sensor according to (2) or (3),
in which the setting information includes a predetermined measurement threshold value, and
the first measurement period is a period from a timing at which the parameter less than the measurement threshold value is measured to the irrigation timing.

(5) The sensor according to any one of (2) to (4),
in which the setting information includes information indicating whether an installation place of the sensor is indoors or outdoors.

(6) The sensor according to any one of (1) to (5),
in which the control unit controls the measurement frequency within a second measurement period immediately after the irrigation timing to a value higher than the predetermined value, and controls the measurement frequency within a period that corresponds to neither the first measurement period nor the second measurement period to a value lower than the predetermined value.

(7) The sensor according to (6),
in which the control unit controls the measurement frequency within a third measurement period immediately after rainfall to a value higher than the predetermined value, and controls the measurement frequency within a period that corresponds to none of the first measurement period, the second measurement period, and the third measurement period to a value lower than the predetermined value.

(8) The sensor according to any one of (1) to (7),
in which the parameter is a water content. (9) The sensor according to any one of (1) to (7),
in which the parameter is a water potential. (10) A control device including:
a communication unit that transmits a control signal to a sensor that measures a parameter related to moisture of soil; and
a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value.

(11) The control device according to (10),
in which the parameter includes a water content and a water potential,
the sensor includes a moisture sensor that measures the water content and a water potential sensor that measures the water potential, and
the control unit causes the moisture sensor to measure a new water content in a case where the measured water content exceeds a predetermined switching threshold value, and causes the water potential sensor to measure the water potential in a case where the measured water content does not exceed the switching threshold value.

(12) A measurement system including:
a sensor that measures a parameter related to moisture of soil; and a control device that includes a communication unit that transmits a control signal to the sensor, and a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value.

(13) A control method of a sensor, the control method including:

a measurement procedure of measuring a parameter related to moisture of soil; and
a control procedure of controlling a measurement frequency of the parameter within a first measurement period immediately before an irrigation timing to the soil to a value higher than a predetermined value, and controlling the measurement frequency before the first measurement period to a value lower than the predetermined value.

(14) A control method of a control device, the control method including:
a communication procedure of transmitting a control signal to a sensor that measures a parameter related to moisture of soil; and
a control procedure of, by the control signal, controlling a measurement frequency of the parameter within a first measurement period immediately before an irrigation timing to the soil to a value higher than a predetermined value, and controlling the measurement frequency before the first measurement period to a value lower than the predetermined value.

(15) A sensor including:
a measurement section that measures a parameter related to moisture of soil; and
a control unit that controls measurement accuracy of the parameter on a basis of predetermined setting information.

(16) A control device including:
a communication unit that transmits a control signal to a sensor that measures a parameter related to moisture of soil; and
a control unit that controls measurement accuracy of the parameter by the control signal on a basis of predetermined setting information.

### REFERENCE SIGNS LIST

110 Weather server
120 Irrigation device
130 External sensor
200 Measurement system
210 User terminal
211 Operation unit
212 Control unit
213 Display unit
214, 221, 311 Communication unit
220 Control device
222 Irrigation/sensor control unit
223, 313 Storage unit
300 Moisture sensor
310 Measurement unit
312 Sensor control unit
320 Measurement section
321 Transmission probe
322 Reception probe
330 Battery
400 Water potential sensor

1. A sensor comprising:
a measurement section that measures a parameter related to moisture of soil; and
a control unit that controls a measurement frequency of the parameter within a first measurement period immediately before an irrigation timing to the soil to a value higher than a predetermined value, and controls the measurement frequency before the first measurement period to a value lower than the predetermined value.

2. The sensor according to claim 1, further comprising:
a communication unit that receives predetermined setting information.

3. The sensor according to claim 2,
wherein the setting information includes the irrigation timing, and
the first measurement period is a period from a timing, which is a predetermined time before the irrigation timing, to the irrigation timing.

4. The sensor according to claim 2,
wherein the setting information includes a predetermined measurement threshold value, and
the first measurement period is a period from a timing at which the parameter less than the measurement threshold value is measured to the irrigation timing.

5. The sensor according to claim 2,
wherein the setting information includes information indicating whether an installation place of the sensor is indoors or outdoors.

6. The sensor according to claim 1,
wherein the control unit controls the measurement frequency within a second measurement period immediately after the irrigation timing to a value higher than the predetermined value, and controls the measurement frequency within a period that corresponds to neither the first measurement period nor the second measurement period to a value lower than the predetermined value.

7. The sensor according to claim 6,
wherein the control unit controls the measurement frequency within a third measurement period immediately after rainfall to a value higher than the predetermined value, and controls the measurement frequency within a period that corresponds to none of the first measurement period, the second measurement period, and the third measurement period to a value lower than the predetermined value.

8. The sensor according to claim 1,
wherein the parameter is a water content.

9. The sensor according to claim 1,
wherein the parameter is a water potential.

10. A control device comprising:
a communication unit that transmits a control signal to a sensor that measures a parameter related to moisture of soil; and
a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value.

11. The control device according to claim 10,
wherein the parameter includes a water content and a water potential,
the sensor includes a moisture sensor that measures the water content and a water potential sensor that measures the water potential, and
the control unit causes the moisture sensor to measure a new water content in a case where the measured water content exceeds a predetermined switching threshold value, and causes the water potential sensor to measure the water potential in a case where the measured water content does not exceed the switching threshold value.

12. A measurement system comprising:
a sensor that measures a parameter related to moisture of soil; and
a control device that includes a communication unit that transmits a control signal to the sensor, and a control unit that, by the control signal, controls a measurement frequency of the parameter within a first measurement period immediately before an irrigation timing to the soil to a value higher than a predetermined value, and controls the measurement frequency before the first measurement period to a value lower than the predetermined value.

13. A control method of a sensor, the control method comprising:
a measurement procedure of measuring a parameter related to moisture of soil; and
a control procedure of controlling a measurement frequency of the parameter within a first measurement period immediately before an irrigation timing to the soil to a value higher than a predetermined value, and controlling the measurement frequency before the first measurement period to a value lower than the predetermined value.

14. A control method of a control device, the control method comprising:
a communication procedure of transmitting a control signal to a sensor that measures a parameter related to moisture of soil; and
a control procedure of, by the control signal, controlling a measurement frequency of the parameter within a first measurement period immediately before an irrigation timing to the soil to a value higher than a predetermined value, and controlling the measurement frequency before the first measurement period to a value lower than the predetermined value.

15. A sensor comprising:
a measurement section that measures a parameter related to moisture of soil; and
a control unit that controls measurement accuracy of the parameter on a basis of predetermined setting information.

16. A control device comprising:
a communication unit that transmits a control signal to a sensor that measures a parameter related to moisture of soil; and
a control unit that controls measurement accuracy of the parameter by the control signal on a basis of predetermined setting information.
